(19) United States
(12) Patent Application Publication
(10) Pub. No.: US 2025/0075337 A1
(43) Pub. Date: Mar. 6, 2025

(54) SATURATED QUATERNARY AMMONIUM COMPOUNDS, GLUTARALDEHYDE, AND TETRAKIS(HYDROXYMETHYL)PHOSPHONIUM SULFATE FOR CORROSION INHIBITION, CLEANING, AND BIOCIDAL EFFICACY IN HYDROGEN SYSTEMS

(71) Applicant: ChampionX LLC, Sugar Land, TX (US)

(72) Inventors: Jeremy Moloney, Katy, TX (US); Jing Wu, Missouri City, TX (US); Shelley Zhou, Sugar Land, TX (US)

(21) Appl. No.: 18/804,570

(22) Filed: Aug. 14, 2024

Related U.S. Application Data
(60) Provisional application No. 63/662,544, filed on Jun. 21, 2024.
(60) Provisional application No. 63/534,907, filed on Aug. 28, 2023.

(51) Int. Cl. C23F 11/06; A61L 2/18; A61L 101/34; C02F 1/50; C02F 5/12; C11D 3/20; C11D 3/30; C11D 3/36; C11D 3/43; C11D 3/48

(52) U.S. Cl. CPC C23F 11/06; A61L 2/18; C02F 1/50; C02F 5/12; C11D 3/2072; C11D 3/30; C11D 3/362; C11D 3/43; C11D 3/48; A61L 2101/34; A61L 2202/17; C02F 2303/04; C02F 2303/08; C02F 2303/20

(57) ABSTRACT

Methods for inhibiting corrosion, cleaning and/or providing biocidal efficacy in hydrogen rich environments that are in hydrogen systems or hydrogen mediums using saturated alkyl diquaternary compound containing compositions, saturated alkyl quaternary compound containing compositions, aldehyde containing compositions, and/or tetrakis(hydroxymethyl)phosphonium sulfate containing compositions are provided. The methods provide efficacious corrosion inhibition, cleaning and/or biocidal efficacy without loss of stability due to hydrogenation in hydrogen rich environments.

### CROSS REFERENCE TO RELATED APPLICATIONS

This application claims priority under 35 U.S.C. § 119 to provisional patent applications U.S. Ser. No. 63/534,907 filed Aug. 28, 2023 and U.S. Ser. No. 63/662,554 filed Jun. 21, 2024. The provisional patent application is herein incorporated by reference in its entirety, including without limitation, the specification, claims, and abstract, as well as any figures, tables, appendices, or drawings thereof.

### TECHNICAL FIELD

The present disclosure relates generally to methods for inhibiting corrosion, cleaning compositions and/or biocidal efficacy in hydrogen rich environments using saturated alkyl diquaternary compound containing compositions, saturated alkyl quaternary compound containing compositions, aldehyde containing compositions, and tetrakis(hydroxymethyl)phosphonium sulfate containing compositions. The methods provide efficacious corrosion inhibition, cleaning and/or biocidal efficacy without loss of stability due to hydrogenation in hydrogen rich environments.

### BACKGROUND

Hydrogen is anticipated to play a critical role in the global energy transition, serving as a clean and versatile energy carrier. Its significance lies in its potential to decarbonize various sectors of the economy. There are multiple sources of hydrogen including those termed green, blue, grey and turquoise. Blue, grey and turquoise are produced from natural gas with carbon capture and storage (CCS) technologies, natural gas without carbon capture and through the gasification of methane coupled with CCS, respectively. Thus, whilst these streams are predominately hydrogen, there is also the presence of other components including carbon dioxide, which is known to be corrosive to carbon steel pipelines, and therefore asset integrity of such infrastructure is important.

The deployment of corrosion inhibitors to mitigate corrosion of carbon steel pipelines is utilized in the oil and gas industry and these are often extended into hydrogen pipelines to provide the same asset integrity. In most applications corrosion inhibitors are used to reduce internal corrosion in metal containments which are contacted by aqueous liquids (including fluids and/or gases) containing corrodents. Corrosion inhibitors are added to the fluids which come into contact with metal surfaces where they act to prevent, retard, delay, reverse, and/or otherwise inhibit the corrosion of metal surfaces such as carbon-steel metal surfaces. However, these corrosion inhibitor chemistries are unsaturated, containing carbon-carbon double bonds, and hydrogenation causes instability and performance issues. In particular, hydrogen often reacts with certain organic molecules to form saturated compounds and many chemicals commonly utilized within the energy sector include molecules that are susceptible to these hydrogenation reactions.

In addition to corrosion remaining a significant challenge in hydrogen systems, there is also the need for cleaning and biocidal efficacy therein due to bacteria and biofouling. Depending on the source and handling of the hydrogen stream, there can be a range of cleanliness levels of the streams based on bacteria and biofouling therein. Biofouling can impede the efficient transportation of hydrogen by reducing pipeline diameter and causing pressure drops. In addition, microbial activities may produce byproducts that can contribute to corrosion, often referred to as microbial influenced corrosion (MIC), compromising the integrity of the pipeline. When MIC is suspected in a system, the main area of concern becomes the biofilm, or sessile organisms, on the surface of the pipeline.

Hydrogen can also react with certain materials in the systems, such as pipelines, leading to formations of solid deposits. Still further, variations in temperature and pressure can influence the deposition of substances within the pipelines.

The deployment of various chemicals as biocides are utilized in the oil and gas industry and these are often extended into hydrogen pipelines to provide the same asset integrity. In most applications biocides are added to the fluids to kill bacteria and prevent biofouling. However, these biocides are often unsaturated, containing carbon-carbon double bonds, and hydrogenation causes instability and performance issues. In particular, hydrogen often reacts with certain organic molecules to form saturated compounds and many chemicals commonly utilized within the energy sector include molecules that are susceptible to these hydrogenation reactions. Various biocidal chemicals are oxidizers that are therefore undesirable in hydrogen systems, such as for example ethylene oxide, propylene oxide, acrolein, 2-butyne-1,4-diol (2,4-DB), triclosan, cinnamaldehyde, silver nanoparticles, ozone, and certain unsaturated quaternary ammonium compounds.

Surfactants that are used as cleaning agents also often include unsaturated carbon bonds as they have both hydrophobic and hydrophilic regions and the hydrophobic portions often include unsaturated carbon bonds. These unsaturated carbon bonds contribute to solvent properties of the cleaning agents making them effective in dissolving certain types of residues, stains, or contaminants. Various surfactants for cleaning have unsaturated carbon bonds that include oxygenated functional groups such as aldehydes, ketones, or esters, or have aromatic structures to aid in ability to dissolve and remove certain types of stains and residues or polyunsaturated compounds to break down complex organic molecules.

Corrosion inhibition, surface and infrastructure cleaning and preventing of biofouling are all beneficial to processing systems in that they extend the lifespan of metal surfaces, as well as permit the use of carbon steel components rather than more expensive metals, such as nickel, cobalt, and chromium alloys or other materials more expensive than carbon steel and/or which inherently entail other disadvantages in suitability for the purpose of fluid containment.

Managing bacteria and biofouling as well as preventing corrosion in hydrogen systems, including pipelines, is crucial to maintain the reliability and safety of the infrastructure for storing and transporting hydrogen gas.

It is an object of this disclosure to provide saturated alkyl diquaternary compounds and saturated alkyl quaternary compounds in methods for inhibiting corrosion, cleaning and preventing biofouling in hydrogen systems.

It is an object of this disclosure to provide glutaraldehyde containing compositions in methods for preventing biofouling in hydrogen systems.

It is an object of this disclosure to provide tetrakis(hydroxymethyl)phosphonium sulfate containing compositions in methods for preventing biofouling in hydrogen systems.

Other objects, embodiments advantages of this disclosure will be apparent to one skilled in the art in view of the following disclosure, the drawings, and the appended claims.

### SUMMARY

The following objects, features, advantages, aspects, and/or embodiments, are not exhaustive and do not limit the overall disclosure. No single embodiment need provide each and every object, feature, or advantage. Any of the objects, features, advantages, aspects, and/or embodiments disclosed herein can be integrated with one another, either in full or in part.

It is an object, feature, and/or advantage of the present disclosure to provide methods for inhibiting corrosion in hydrogen systems, namely pipelines, containing hydrogen.

In a particular aspect, methods of inhibiting corrosion in a hydrogen system comprise: contacting a surface comprising metal in a hydrogen system or medium with a corrosion-inhibiting composition comprising a saturated alkyl diquaternary ammonium compound having the following structure (III as described herein) or a saturated alkyl quaternary ammonium compound having the following structure (IV as described herein); and reducing corrosion on the surface.

It is a further object, feature, and/or advantage of the present disclosure to provide methods for cleaning and/or inhibiting or removing biofouling in hydrogen systems, namely pipelines, containing hydrogen.

In a particular aspect, methods of cleaning and/or preventing biofouling in a hydrogen system comprise: contacting a surface in a hydrogen system or medium with a cleaning and/or biocidal composition comprising a saturated alkyl diquaternary ammonium compound, a saturated alkyl quaternary ammonium compound, an aldehyde comprising glutaraldehyde, formaldehyde or glyoxal, and/or tetrakis(hydroxymethyl)phosphonium sulfate; wherein the saturated alkyl diquaternary ammonium compound has the structure (III as described herein) or a saturated alkyl quaternary ammonium compound having the following structure (IV as described herein).

These and/or other objects, features, advantages, aspects, and/or embodiments will become apparent to those skilled in the art after reviewing the following brief and detailed descriptions of the drawings. Furthermore, the present disclosure encompasses aspects and/or embodiments not expressly disclosed but which can be understood from a reading of the present disclosure, including at least: (a) combinations of disclosed aspects and/or embodiments and/or (b) reasonable modifications not shown or described.

While multiple embodiments are disclosed, still other embodiments will become apparent to those skilled in the art from the following detailed description, which shows and describes illustrative embodiments. Accordingly, the drawings and detailed description are to be regarded as illustrative in nature and not restrictive.

### BRIEF DESCRIPTION OF THE DRAWINGS

FIG. 1 shows a reaction to produce the alkyl diquaternary compounds of structure (III) as described herein.

FIG. 2 shows a further reaction to produce the alkyl diquaternary compounds of structure (IIIa) as described herein.

Various embodiments of the present disclosure will be described in detail with reference to the drawings, wherein like reference numerals represent like parts throughout the several views. Reference to various embodiments does not limit the scope of the disclosure. Figures represented herein are not limitations to the various embodiments according to the disclosure and are presented for exemplary illustration of the invention. An artisan of ordinary skill in the art need not view, within isolated figure(s), the near infinite number of distinct permutations of features described in the following detailed description to facilitate an understanding of the present invention.

### DETAILED DESCRIPTION

The present disclosure is not to be limited to that described herein, which can vary and are understood by skilled artisans. No features shown or described are essential to permit basic operation of the present disclosure unless otherwise indicated. It has been beneficially found that saturated diquaternary ammonium compounds and saturated quaternary ammonium compounds as described herein provide effective corrosion-inhibiting, cleaning and biocidal efficacy in treating surfaces within hydrogen systems. It has been further beneficially found that glutaraldehyde and/or tetrakis(hydroxymethyl)phosphonium sulfate compounds as described herein provide effective biocidal efficacy in treating surfaces within hydrogen systems.

It is further to be understood that all terminology used herein is for the purpose of describing particular embodiments only, and is not intended to be limiting in any manner or scope. For example, as used in this specification and the appended claims, the singular forms “a,” “an” and “the” can include plural referents unless the content clearly indicates otherwise. Further, all units, prefixes, and symbols may be denoted in its SI accepted form.

Numeric ranges recited within the specification are inclusive of the numbers defining the range and include each integer within the defined range. Throughout this disclosure, various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4%. This applies regardless of the breadth of the range.

As used herein, the term “and/or”, e.g., “X and/or Y” shall be understood to mean either “X and Y” or “X or Y” and shall be taken to provide explicit support for both meanings or for either meaning, e.g. A and/or B includes the options i) A, ii) B or iii) A and B.

It is to be appreciated that certain features that are, for clarity, described herein in the context of separate embodiments, may also be provided in combination in a single embodiment. Conversely, various features that are, for brevity, described in the context of a single embodiment, may also be provided separately or in any sub-combination.

The methods of the present disclosure may comprise, consist essentially of, or consist of the components and ingredients of the present disclosure as well as other ingredients described herein. As used herein, “consisting essentially of” means that the methods, systems, apparatuses and compositions may include additional steps, components or ingredients, but only if the additional steps, components or ingredients do not materially alter the basic and novel characteristics of the claimed methods, systems, apparatuses, and compositions.

Unless defined otherwise, all technical and scientific terms used above have the same meaning as commonly understood by one of ordinary skill in the art to which embodiments of the present disclosure pertain.

The terms “invention” or “present invention” are not intended to refer to any single embodiment of the particular invention but encompass all possible embodiments as described in the specification and the claims.

The term “about,” as used herein, refers to variation in the numerical quantity that can occur, for example, through typical measuring techniques and equipment, with respect to any quantifiable variable, including, but not limited to, concentration, mass, volume, time, molecular weight, temperature, pH, molar ratios, log reduction, and the like. The term “about” also encompasses these variations. Whether or not modified by the term “about,” the claims include equivalents to the quantities.

As used herein, the term “alkyl” or “alkyl groups” refers to saturated hydrocarbons having one or more carbon atoms, including straight-chain alkyl groups (e.g., methyl, ethyl, propyl, butyl, pentyl, hexyl, heptyl, octyl, nonyl, decyl, etc.), cyclic alkyl groups (or “cycloalkyl” or “alicyclic” or “carbocyclic” groups) (e.g., cyclopropyl, cyclopentyl, cyclohexyl, cycloheptyl, cyclooctyl, etc.), branched-chain alkyl groups (e.g., isopropyl, tert-butyl, sec-butyl, isobutyl, etc.), and alkyl-substituted alkyl groups (e.g., alkyl-substituted cycloalkyl groups and cycloalkyl-substituted alkyl groups). Unless otherwise specified, the term “alkyl” includes both “unsubstituted alkyls” and “substituted alkyls.” As used herein, the term “substituted alkyls” refers to alkyl groups having substituents replacing one or more hydrogens on one or more carbons of the hydrocarbon backbone. Such substituents may include, for example, alkenyl, alkynyl, halogeno, hydroxyl, alkylcarbonyloxy, arylcarbonyloxy, alkoxycarbonyloxy, aryloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), imino, sulfhydryl, alkylthio, arylthio, thiocarboxylate, sulfates, alkylsulfinyl, sulfonates, sulfamoyl, sulfonamido, nitro, trifluoromethyl, cyano, azido, heterocyclic, alkylaryl, or aromatic (including heteroaromatic) groups.

As used herein, the term “between” is inclusive of any endpoints noted relative to a described range.

As used herein, the term “containment” or “metal containment” includes any metal surface or portion thereof that is in contact with fluids in a hydrogen system. In embodiments the containment is in fluid communication with one or more devices or apparatuses, including other containments. In embodiments the containment is a pipe. In embodiments the containment is a tank. In embodiments, the metal is steel. In embodiments, the steel is carbon steel. In embodiments, the carbon steel is stainless steel.

As used herein, the term “corrodent” refers to one or more salts and/or other dissolved solids, liquids, or gasses that cause, accelerate, or promote corrosion. Non-limiting examples of corrodents are oxygen, hydrogen sulfide, hydrogen chloride, carbon dioxide, sodium chloride, calcium chloride, sulfur dioxide, and combinations thereof. In exemplary embodiments, corrodents are capable of corroding a carbon steel at a rate of at least about 100 milli-inches per year (mpy).

The term “-ene” as used as a suffix as part of another group denotes a bivalent substituent in which a hydrogen atom is removed from each of two terminal carbons of the group, or if the group is cyclic, from each of two different carbon atoms in the ring. For example, alkylene denotes a bivalent alkyl group such as methylene (—CH₂—) or ethylene (—CH₂CH₂—), and arylene denotes a bivalent aryl group such as o-phenylene, m-phenylene, or p-phenylene.

As used herein, the term “exemplary” refers to an example, an instance, or an illustration, and does not indicate a most preferred embodiment unless otherwise stated.

The phrase “free of” or similar phrases if used herein means that the composition comprises 0% of the stated component and refers to a composition where the component has not been intentionally added. However, it will be appreciated that such components may incidentally form thereafter, under some circumstances, or such component may be incidentally present, e.g., as an incidental contaminant.

The term “hydrogen system” as referred to herein includes any system where hydrogen gas is present and in contact with a surface and/or medium, wherein the medium can comprise hydrogen gas, natural gas, hydrogen sulfide gas, ammonia, carbon dioxide (such as in CCS), or any combination thereof.

The term “inhibiting” as referred to herein includes both inhibiting and preventing corrosion on a surface or within a system, namely a hydrogen system.

As used herein, the term “optional” or “optionally” means that the subsequently described component, event or circumstance may but need not be present or occur. The description therefore discloses and includes instances in which the event or circumstance occurs and instances in which it does not, or instances in which the described component is present and instances in which it is not.

As used herein the term “polymer” refers to a molecular complex comprised of a more than ten monomeric units and generally includes, but is not limited to, homopolymers, copolymers, such as for example, block, graft, random and alternating copolymers, terpolymers, and higher “x”mers, further including their analogs, derivatives, combinations, and blends thereof. Furthermore, unless otherwise specifically limited, the term “polymer” shall include all possible isomeric configurations of the molecule, including, but are not limited to isotactic, syndiotactic and random symmetries, and combinations thereof. Furthermore, unless otherwise specifically limited, the term “polymer” shall include all possible geometrical configurations of the molecule.

The “scope” of the present disclosure is defined by the appended claims, along with the full scope of equivalents to which such claims are entitled. The scope of the disclosure is further qualified as including any possible modification to any of the aspects and/or embodiments disclosed herein which would result in other embodiments, combinations, subcombinations, or the like that would be obvious to those skilled in the art.

The term “substituted” as in “substituted aryl,” “substituted alkyl,” and the like, means that in the group in question (i.e., the alkyl, aryl or other group that follows the term), at least one hydrogen atom bound to a carbon atom is replaced with one or more substituent groups such as hydroxy (—OH), alkylthio, phosphino, amido (—CON(R_{A})(R_{B}), wherein R_{A} and R_{B} are independently hydrogen, alkyl, or aryl), amino(—N(R_{A})(R_{B}), wherein R_{A} and R_{B} are independently hydrogen, alkyl, or aryl), halo (fluoro, chloro, bromo, or iodo), silyl, nitro (—NO₂), an ether (—OR_{A} wherein R_{A} is alkyl or aryl), an ester (—OC(O)R_{A} wherein R_{A} is alkyl or aryl), keto (—C(O)R_{A} wherein R_{A} is alkyl or aryl), heterocyclo, and the like. Further, an alkylene group in the chain can be replaced with an ether, an amine, an amide, a carbonyl, an ester, a cycloalkyl, or a heterocyclo functional group. When the term “substituted” introduces a list of possible substituted groups, it is intended that the term apply to every member of that group. That is, the phrase “optionally substituted alkyl or aryl” is to be interpreted as “optionally substituted alkyl or optionally substituted aryl.”

The term “weight percent,” “wt-%,” “percent by weight,” “% by weight,” and variations thereof, as used herein, refer to the concentration of a substance as the weight of that substance divided by the total weight of the composition and multiplied by 100. It is understood that, as used here, “percent,” “%,” and the like are intended to be synonymous with “weight percent,” “wt-%,” etc.

### Saturated Quaternary Ammonium Compounds

According to embodiments, methods of inhibiting corrosion, cleaning and/or providing biocidal efficacy employ an alkyl quaternary ammonium compound and/or an alkyl diquaternary ammonium compound. In embodiments the compositions are solutions or dispersions. The quaternary ammonium compounds can be in a solution or dispersion in a solvent and/or include additional functional components for the corrosion-inhibition, cleaning and/or biocidal efficacy.

The saturated alkyl quaternary ammonium and diquaternary ammonium compounds described herein provide chain lengths for water solubility and hydrophobicity while the two nitrogen heteroatoms provided efficient binding to surfaces in need of corrosion inhibition.

In embodiments employing a saturated alkyl diquaternary ammonium compound the dual nitrogen heteroatoms provide enhanced binding to surfaces to anchor the corrosion-inhibitors to the surfaces including under high shear conditions.

### Saturated Alkyl Diquaternary Compounds

The saturated alkyl diquaternary compounds included in the corrosion-inhibiting compositions have the following structure (III):

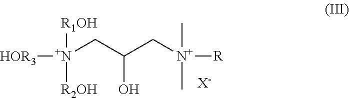

wherein:
R is a linear C₈-C₃₀ alkyl group,
R₁, R₂, and R₃ are independently a linear C₁-C₃ alkyl group, and X— is a halide.

In embodiments, the saturated alkyl diquaternary compounds have the structure (III) wherein:
R is a linear C₈-C₃₀ alkyl group,
R₁, R₂, and R₃ are independently a linear C₁-C₃ alkyl group, and
X— is a halide.

In further embodiments the saturated alkyl diquaternary compounds have the structure (III) wherein:
R is a linear C₁₀-C₂₄ alkyl group,
R₁, R₂, and R₃ are the same linear C₁-C₃ alkyl group, and
X— is a halide, preferably chloro or bromo (Cl^{—} or Br^{—}).

In further embodiments the saturated alkyl diquaternary compounds have the structure (III) wherein:
R is a linear C₁₂-C₂₄ alkyl group,
R₁, R₂, and R₃ are each the same C₁-C₃ alkyl group, or preferably are each C₂ alkyl group, and
X— is chloro or bromo (Cl^{—} or Br^{—}).

In embodiments the alkyl diquaternary compounds have the structure (III) are a quaternization reaction product of

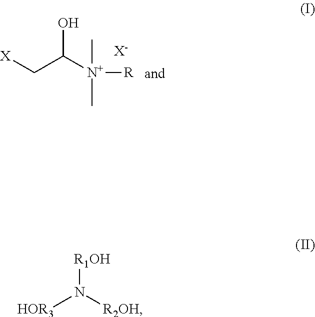

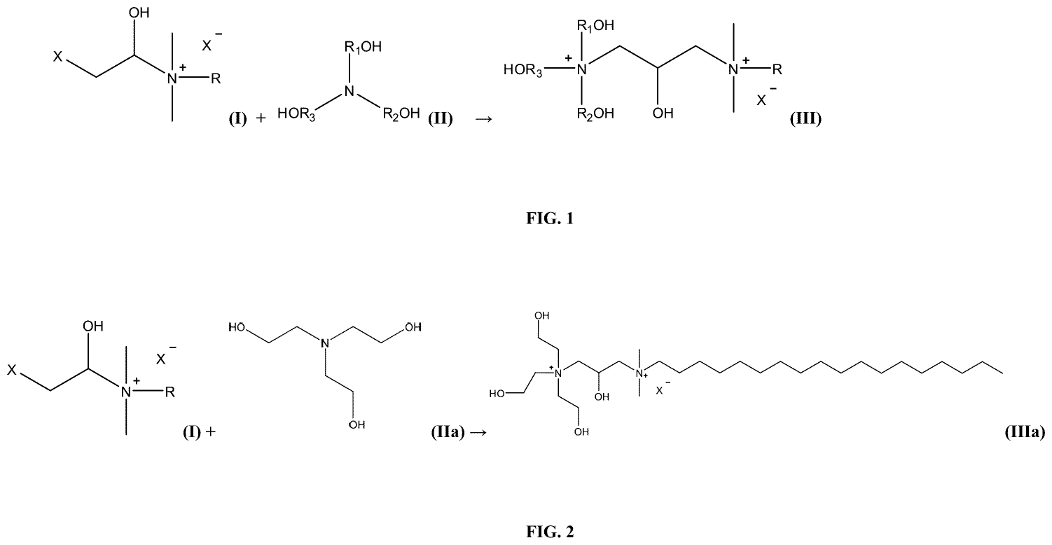

hydroxide source in a quaternizing reaction as shown schematically in FIG. 1. The quaternization reaction can include a solvent, e.g. propylene glycol and water, at an alkaline pH, e.g. pH 9 or greater, at a suitable temperature range, e.g. 85° C.-90° C. The reagent I has the structure as shown wherein R is a linear C₈-C₃₀ alkyl group, X is a halogen and X— is a halide. In preferred embodiments R is a linear C₈-C₃₀, C₁₀-C₂₄, or C₁₂-C₂₄ alkyl group. In preferred embodiments R is a lauryl, cocoalkyl, or stearyl group. Preferred halogens include Cl and Br, and preferred halides are the corresponding Cl^{—} and Br^{—}. The reagent II has the structure as shown wherein R₁, R₂, and R₃ are independently a linear C₁-C₃ alkyl group, R₁, R₂, and R₃ are the same linear C₁-C₃ alkyl group, or R₁, R₂, and R₃ are each C₂ alkyl group. In embodiments the reaction between I and II to make the saturated alkyl diquaternary compounds have the structure (III) have a molar ratio of the two reagents that are about 1:1.

In embodiments the reagent I is a hydrophobically modified chlorohydrin. In embodiments the hydrophobically modified chlorohydrin has an alkyl R group that is from C₈-C₃₀, such as 3-chloro-2-hydroxypropyl-dimethylstearylammonium chloride, commercially available as Quab 426 sold as a 40% solution in a 1,2-propanediol/water mixture.

In still further embodiments the saturated alkyl diquaternary compounds of structure (III) has the following structure:

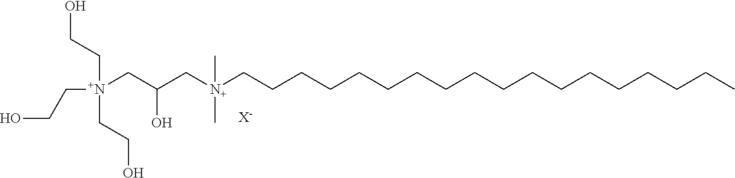

(IIIa) wherein X is Cl— or Br— made in a quaternizing reaction as shown schematically in FIG. 2 formed through the reaction of reagent I and IIa. In embodiments the structure of the alkyl diquaternary compounds of structure (IIIa) having multiple hydroxyl groups aid in water solubility of the corrosion inhibitor and in water partitioning. Without being limited to a particular mechanism of action, the structure (VI) having multiple hydroxyl groups improve the water partitioning of the compound for use in the corrosion-inhibiting compositions. In addition, the alkyl diquaternary compounds with multiple hydroxyl groups further improve water partitioning of products while also controlling corrosion. The water partitioning is improved with the presence of hydroxyl groups due to increased water solubility that increases the partitioning to the water phase resulting in a greater chemical concentration in the water phase available to interact with the metal surface and, in turn, increases the corrosion inhibition.

### Saturated Alkyl Quaternary Compounds

The saturated alkyl quaternary compounds included in the following structure:

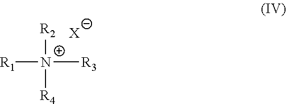

wherein:
R₁, R₂, R₃ and R₄ are independently a linear C₁₋₂₄ alkyl group, and X— is a halide.

In embodiments, the saturated alkyl quaternary compounds have the structure (IV) wherein: R₁, R₂, R₃ and R₄ are independently a linear C₁₀₋₂₄, C₁₀₋₁₈, C₁₀₋₁₆, C₁₀₋₁₄, or C₁₀₋₁₂ alkyl group, and X— is a halide.

In embodiments, the saturated alkyl quaternary compounds have the structure (IV) wherein: R₁ and R₂ are methyl groups, and wherein R₃ and R₄ are independently a linear C₁₀₋₂₄, C₁₀₋₁₈, or C₁₀₋₁₆, alkyl group, and X— is a halide.

### Aldehydes—Glutaraldehyde

According to embodiments, methods of cleaning and providing biocidal efficacy, including controlling biofouling and biofilm, employ an aldehyde comprising glutaraldehyde (C₅H₈O₂ CAS 111-30-8), formaldehyde (CH₂O CAS 50-00-0) or glyoxal (C₂H₂O₂ CAS 107-22-2).

According to preferred embodiments, methods of cleaning and providing biocidal efficacy, including controlling biofouling and biofilm, employ glutaraldehyde. Glutaraldehyde does not contain unsaturated carbon (C═C or C≡C), it is an aliphatic dialdehyde with two adjacent carbonyl (C═O) groups. As a result, there is no hydrogenation with the addition of hydrogen to unsaturated compounds (which are those containing carbon-carbon double or triple bonds). The structure provides the ability to crosslink and denature proteins, disrupting microbial cell structures and functions. Beneficially, the absence of carbon-carbon double or triple bonds in the structures of glutaraldehyde means that it is not susceptible to typical hydrogenation reactions as the bond dissociation energy (BDE) of the carbon-carbon bonds are lower than the carbon-oxygen double bonds. Moreover the carbon-oxygen double bonds also have a higher degree of polarization due to the electronegative oxygen atom as well as steric hindrance. As a result, the unsaturated carbons are susceptible to cleavage by hydrogen.

In embodiments the compositions are solutions or dispersions containing an aldehyde, preferably glutaraldehyde. The aldehyde, preferably glutaraldehyde can be in a solution or dispersion in a solvent and/or include additional functional components for the cleaning, biofouling control and/or biocidal efficacy.

### Tetrakis(Hydroxymethyl)Phosphonium Sulfate

According to embodiments, methods of cleaning and providing biocidal efficacy, including controlling biofouling and biofilm, employ tetrakis(hydroxymethyl)phosphonium sulfate (THPS) (C₈H₂₄O₁₂P₂S CAS 55566-30-8). THPS does not contain unsaturated carbon (C═C or C≡C) and instead has phosphorus-oxygen bonds and is therefore not susceptible to cleavage by hydrogen. THPS is effective against a broad spectrum of microorganisms, and its biocidal activity is related to its ability to interfere with cellular processes.

In embodiments the compositions are solutions or dispersions containing tetrakis(hydroxymethyl)phosphonium sulfate. The tetrakis(hydroxymethyl)phosphonium sulfate can be in a solution or dispersion in a solvent and/or include additional functional components for the cleaning, biofouling control and/or biocidal efficacy.

### Compositions

In embodiments, the various corrosion inhibitors including the saturated alkyl diquaternary compound corrosion inhibitor and/or saturated alkyl quaternary compound corrosion inhibitors can be provided in a corrosion-inhibiting composition at an amount of at least about 10 wt-% to about 70 wt-%, about 10 wt-% to about 60 wt-%, about 20 wt-% to about 60 wt-%, about 20 wt-% to about 50 wt-%, about 25 wt-% to about 50 wt-%, or about 30 wt-% to about 50 wt-%. In addition, without being limited according to the disclosure, all ranges recited are inclusive of the numbers defining the range and include each integer within the defined range.

In embodiments, the various cleaning and/or biocidal compounds, including the glutaraldehyde and/or tetrakis(hydroxymethyl)phosphonium sulfate containing compositions can be provided in an amount of at least about 1 wt-% to about 70 wt-%, about 10 wt-% to about 70 wt-%, about 20 wt-% to about 60 wt-%, or about 20 wt-% to about 50 wt-%. In addition, without being limited according to the disclosure, all ranges recited are inclusive of the numbers defining the range and include each integer within the defined range.

The various compositions for corrosion-inhibition, cleaning and/or biocidal efficacy can be provided with one or more solvents and/or additional functional ingredients.

### Solvent

The chemistries and compositions can be delivered with a solvent. In some embodiments at least one solvent is included in the composition. Exemplary solvents include water, organic solvents and/or aromatic solvents.

Exemplary organic solvents can include an alcohol, a hydrocarbon, a ketone, an ether, an alkylene glycol, a glycol ether, an amide, a nitrile, a sulfoxide, an ester, or a combination thereof. Examples of suitable organic solvents include, but are not limited to, methanol, ethanol, propanol, isopropanol (isopropyl alcohol or 2-propanol), butanol, 2-ethylhexanol, hexanol, octanol, decanol, 2-butoxyethanol, glycols and derivatives (ethylene glycol, methylene glycol, 1,2-propylene glycol, 1,3-propylene glycol, diethyleneglycol monomethyl ether, ethylene glycol monobutyl ether, ethylene glycol dibutyl ether, etc.), pentane, hexane, cyclohexane, methylcyclohexane, heptane, decane, dodecane, diesel, toluene, xylene, heavy aromatic naphtha, cyclohexanone, diisobutylketone, diethyl ether, propylene carbonate, N-methylpyrrolidinone, N,N-dimethylformamide, or a combination thereof.

In some embodiments the solvent is one or more of ethylene glycol, propylene glycol, isopropanol, methanol, alkyl alcohol, and/or water.

In some embodiments, an alcohol solvent is used, including straight chain or branched aliphatic such as methanol, ethanol, propanol, isopropanol, butanol, 2-ethylhexanol, hexanol, octanol, decanol, 2-butoxyethanol, etc.

In some embodiments, the composition comprises one or more solvents selected from the group consisting of isopropanol, methanol, ethanol, 2-ethylhexanol, heavy aromatic naphtha, toluene, ethylene glycol, ethylene glycol monobutyl ether (EGMBE), diethylene glycol monoethyl ether, xylene, aromatic solvents, or any combination thereof.

Exemplary aromatic solvents comprise aromatic hydrocarbons such as toluene, xylene, heavy aromatic naphtha, C9-C11 aromatic hydrocarbons (Aromatic 150 or Aromatic Solvent C10) or a combination thereof. Preferably, the aromatic solvent comprises heavy aromatic naphtha or xylene. In any of the embodiments described the aromatic solvent(s) is preferably combined with water.

In some embodiments, the solvent(s) is included in a composition at an amount of at least about 20 wt-% to about 80 wt-%, about 30 wt-% to about 80 wt-%, about 40 wt-% to about 80 wt-%, about 45 wt-% to about 80 wt-%, or about 50 wt-% to about 80 wt-%. In addition, without being limited according to the disclosure, all ranges recited are inclusive of the numbers defining the range and include each integer within the defined range.

### Additional Functional Ingredients

The composition can further be combined with various functional components suitable for uses disclosed herein. In some embodiments, the compositions include the corrosion inhibitor chemistry, cleaning and/or biocidal chemistry and at least one solvent make up a large amount, or even substantially all of the total weight of the compositions. In some embodiments, the corrosion inhibitor chemistry, cleaning and/or biocidal chemistry and at least one solvent, further include an additional functional ingredient selected from the group consisting of synergist, additional corrosion inhibitor, surfactants, polymers, pH modifiers, asphaltene inhibitor, paraffin inhibitor, scale inhibitor, chelant, emulsifier, emulsion breaker, water clarifier, dispersant, or combinations thereof make up a large amount, or even substantially all of the total weight of the compositions. For example, in some embodiments few or no additional functional ingredients are disposed therein.

In other embodiments, additional functional ingredients may be included. The functional ingredients provide desired properties and functionalities to the compositions. For the purpose of this application, the term “functional ingredient” includes a material that when dispersed or dissolved in a use and/or concentrate solution, such as an aqueous solution, provides a beneficial property in a particular use. Some particular examples of functional materials are discussed in more detail below, although the particular materials discussed are given by way of example only, and that a broad variety of other functional ingredients may be used. For example, many of the functional materials discussed below relate to materials used in cleaning. However, other embodiments may include functional ingredients for use in other applications.

In some embodiments, the compositions may include synergist, additional corrosion inhibitors, solvents, pH modifiers, surfactants, hydrate inhibitors, scale inhibitors, biocides, salt substitutes, relative permeability modifiers, sulfide scavengers, breakers, asphaltene inhibitors, paraffin inhibitors, metal complexing agents (chelants), emulsifiers, demulsifiers, iron control agents, friction reducers, drag reducing agents, flow improvers, viscosity reducers, and the like. Exemplary types of the various additional functional ingredients are included in U.S. Pat. No. 11,242,480, which is incorporated by reference in its disclosure of the various listings of additional functional ingredients.

In an embodiment the compositions include a non-emulsifier and/or synergist. Non-emulsifiers include but are not limited to polyethers, oxyalkylates, oxyalkylated polymers, oxyalkylated resins, or combinations thereof.

According to embodiments of the disclosure, the combination of any additional functional ingredients may be provided in a composition in the amount from about 0 wt-% and about 50 wt-%, from about 1 wt-% and about 50 wt-%, or from about 1 wt-% and about 20 wt-%. In addition, without being limited according to the disclosure, all ranges recited are inclusive of the numbers defining the range and include each integer within the defined range.

### Methods of Use

The methods of use described herein for corrosion inhibition and cleaning and/or biocidal efficacy are suitable for use in hydrogen systems or mediums. As referred to herein the hydrogen systems or mediums comprises hydrogen gas, natural gas, hydrogen sulfide gas, ammonia, or a combination thereof. In some embodiments the hydrogen medium comprises hydrogen gas and one or more of methane, ethane, propane, nitrogen, and/or carbon dioxide.

The hydrogen referred to herein may be obtained from many different sources or processes, and may further include combinations, such as mixed streams of hydrogen. For example, “green hydrogen” is hydrogen produced through a water electrolysis process. Carbon dioxide is not emitted during the process. Electricity is used to decompose water into oxygen and hydrogen gas. While “blue hydrogen” may be sourced from fossil fuel, carbon dioxide produced during the process is captured and stored underground, thereby making the overall process carbon neutral. As an additional example, “pink hydrogen” is generated through the electrolysis of water using electricity from a nuclear power plant. Again, the source of the hydrogen to be used in accordance with the present disclosure is not limited so any type of hydrogen or mixed streams with combinations thereof, may be used in accordance with the present disclosure, such as green, blue, pink, gray, black, brown, turquoise, purple, white, red, or where it is generated in the value chain.

Hydrogen systems and mediums can include a wet gas, a dry gas, a dry gas comprising a gas condensate, a wet gas comprising a gas condensate, a wet gas comprising water and a gas condensate, an aqueous medium, a non-aqueous medium, an organic medium, a gaseous medium, and any combination thereof.

In embodiments the hydrogen system or medium can include a hydrogen gas, natural gas, hydrogen sulfide gas, ammonia, or a combination thereof. In some embodiments, the hydrogen medium comprises hydrogen gas and one or more of methane, ethane, propane, nitrogen, and/or carbon dioxide.

The methods are useful in treating surfaces comprising metal in a hydrogen system or medium. In various embodiments the surface is a containment used in the production, transportation (including onshore and offshore hydrogen transportation), storage and/or separation of hydrogen gas.

The hydrogen systems can include hydrogen pipelines, oil and gas pipelines and refineries, including for example, pipelines, storage and/or transportation tanks, distribution systems and the like.

The system includes a fluid to which the composition a chemical is added. A fluid to which the compositions can be introduced can be a hydrogen gas, a hydrocarbon fluid or gas, or combination thereof. The fluid can be contained in and/or exposed to many different types of apparatuses. In embodiments, the fluid is contained in a containment, such as a hydrogen pipeline, storage and/or transportation tanks, distribution systems and the like.

The compositions can be applied by any appropriate method for ensuring dispersal through the fluid. The compositions can be applied to a fluid using various well-known methods and they can be applied at numerous different locations throughout a given system. For example, the compositions can be injected using mechanical equipment such as chemical injection pumps, piping tees, injection fittings, atomizers, quills, and the like. The compositions can be pumped into an oil and/or gas pipeline using an umbilical line. A capillary injection system can be used to deliver the compounds/compositions to a selected fluid.

The compositions can be added to the system by any suitable means, including for example by injecting the composition into a fluid or gas in contact with the metal surface, pumping the composition onto the metal surface, pouring the composition onto the metal surface, spraying the composition onto the metal surface, wiping the metal surface with the composition, coating the metal surface with the composition, dipping the metal surface in the composition, soaking the metal surface in the composition, or any combination thereof.

### Methods of Corrosion Inhibition

The saturated alkyl diquaternary ammonium compounds and/or the saturated alkyl quaternary ammonium compounds are useful in providing corrosion-inhibition to a system in need of effective corrosion control. The methods are suitable for controlling both general and localized corrosion as well as reducing pitting on surfaces.

The saturated alkyl diquaternary ammonium compounds and/or the saturated alkyl quaternary ammonium compounds can be provided in a single composition to a hydrogen system or medium or can be combined with other components, such as solvents, synergists, additional corrosion inhibitors and/or other additional functional ingredients, and the like. In referring to compositions, the scope of the methods of use disclosure also includes combining more than one input (i.e. composition) for the treatment of a hydrogen system or medium.

The methods of use include adding a corrosive inhibiting effective amount of the corrosion-inhibiting compositions to a hydrogen system or medium having at least one metal surface and inhibiting corrosion of the at least one surface. Beneficially, the composition reduces corrosion of a surface compared to a corrosive environment that does not contain the saturated alkyl diquaternary ammonium or saturated alkyl quaternary ammonium compounds. For example, the composition provides improved or at least the same corrosion inhibition of a surface compared to a corrosive environment treated with a conventional quaternary ammonium chloride corrosion inhibitor (e.g. alkyl dimethyl benzyl chloride quat). In embodiments, the corrosion reduction (i.e. inhibition efficiency) is at least about 80%, at least about 90%, at least about 95%, or at least about 99% for a surface comprising metal.

The methods apply the corrosion-inhibiting compositions to a system or medium in need of preventing, reducing or mitigating corrosion. Beneficially, for the corrosion inhibition both localized and generalized corrosion are reduced, as measured by mils penetration per year or milli-inch (one thousandth of an inch) (MPY). MPY is used as an estimated general corrosion rate. The MPY is calculated from the following equation:

$$MPY = ( Δ M [ g ] \cdot C ) / ( ρ [ \frac{g}{{cm}^{2}} ] \cdot A [ {in}^{2} ] \cdot t [ hr ] )$$

where ΔM is the mass loss of the coupon at the end of the test in grams, C is a constant equal to 534000, ρ is the density of the coupon in g/cm², A is the surface area of the coupon in cm², and t is the exposure time in hours.

The method comprises adding the corrosion-inhibiting compositions in a corrosive inhibiting effective amount. The dosage amounts of the compositions described herein to be added to a hydrogen system or medium can be tailored by one skilled in the art based on factors for each system, including, for example, content of fluid (including gas), volume of the fluid (including gas), surface area of the system, temperatures, pH, and CO₂ content. In embodiments, an effective amount of the composition is from about 1 ppm to about 5,000 ppm, based on the total volume of the system. In embodiments, an effective amount of the composition is from about from about 1 ppm to about 1,000 ppm, based on the total volume of the system. In embodiments, an effective amount of the composition is from about from about 10 ppm to about 1,000 ppm, based on the total volume of the system. In further embodiments, an effective amount of the composition is from about from about 10 ppm to about 100 ppm, based on the total volume of the system.

The compositions can be added to the system manually or automatically in either a batch or continuous manner to provide the effective amount of the corrosion-inhibiting composition. In some embodiments, the compositions (or in further embodiments the saturated dialkyl quaternary ammonium compound or the saturated alkyl quaternary ammonium compound in the corrosion-inhibiting compositions) are added to a flow line to provide a corrosive inhibiting effective amount from about 1 to about 5,000 parts per million (ppm), about 1 ppm to about 1,000 ppm, or about 10 ppm to about 1,000 ppm, based on the total volume of the system. In some embodiments, the compositions are added to a flow line to provide a corrosive inhibiting effective amount from at least about 1 ppm, 2 ppm, 5 ppm, 10 ppm, 20 ppm, 50 ppm, 100 ppm, 200 ppm, 250 ppm, 500 ppm, or 1,000 ppm based on the total volume of the system. Each system can have its own dose level requirements, and the effective dose level of the composition to sufficiently reduce the rate of corrosion can vary with the system in which it is used.

The compositions are applied to fluids in a hydrogen system at varying pH ranges. In an embodiment the pH of the fluids will be between about 2 and about 8.

The hydrogen systems comprise a metal surface in need of corrosion inhibition. Exemplary surfaces can include separation vessels, dehydration units, gas lines, oil and/or gas pipelines, or other part of an oil and/or gas refinery. Similarly, the fluid can be contained in and/or exposed to an apparatus used in a hydrogen system. The apparatus can be a cargo vessel, a storage vessel, a holding tank, or a pipeline connecting the tanks, vessels, or processing units.

The system comprises a metal surface subject to corrosion. In embodiments the surfaces can include a variety of metal surfaces that are subject to corrosion. The metals can comprise a component selected from the group consisting of mild steel, galvanized steel, carbon steel, aluminum, aluminum alloys, copper, copper nickel alloys, copper zinc alloys, brass, chrome steels, ferritic alloy steels, austenitic stainless steels, precipitation-hardened stainless steels, high nickel content steels, and any combination thereof.

The compositions are particularly well suited for corrosion inhibition in harsh conditions, including temperatures, flow line conditions (e.g. low and high shear stress, e.g. >50 Pa), high water cut (e.g. >20%), and the like.

In embodiments, treated metal containments are provided with the methods described herein for methods of inhibiting corrosion. In embodiments a metal containment comprises a metal surface and a barrier or film substantially coating the metal surface with a corrosive inhibiting effective amount of the compositions (or the saturated alkyl diquaternary ammonium compounds and/or the saturated alkyl quaternary ammonium compounds corrosion inhibitors described herein).

The compositions can be applied to a fluid in a hydrogen system at any selected temperature, such as ambient temperature or an elevated temperature. The fluid (e.g., hydrogen gas) can be at a temperature of from about −250° C. to about 300° C. (including cryogenic conditions), or from about −250° C. to about 250° C., or most often between about −20° C. to about 50° C.

### Methods of Biofouling Control

The saturated alkyl quaternary ammonium compound containing compositions, aldehyde containing compositions, namely glutaraldehyde containing compositions, and/or tetrakis(hydroxymethyl)phosphonium sulfate containing compositions are useful in providing biofouling control or biocidal efficacy to a system in need thereof. The methods are suitable for controlling microbial populations within hydrogen systems to prevent contamination and system fouling.

Bacteria and biofouling in hydrogen systems, including hydrogen pipelines refer to the presence and growth of microorganisms, such as bacteria, in the pipelines transporting hydrogen gas. Biofouling is the accumulation of organic materials, including microbial growth, on the surfaces such as pipeline surfaces. Moreover, biofouling involves the formation of biofilms, which are layers of microorganisms and extracellular substances that adhere to the pipeline surfaces. Biofouling can lead to the formation of deposits, affecting the flow of hydrogen and potentially causing corrosion. For example, bacteria can exist in hydrogen pipelines due to the presence of trace impurities or residual moisture in the gas. Some bacteria have the ability to metabolize hydrogen and may thrive in pipeline environments.

The saturated alkyl quaternary ammonium compound containing compositions, aldehyde containing compositions, namely glutaraldehyde containing compositions, and/or tetrakis(hydroxymethyl)phosphonium sulfate containing compositions can be provided in a single composition to a hydrogen system or medium or can be combined with other components, such as solvents, synergists, corrosion inhibitors, additional biocides and/or other additional functional ingredients, and the like. In referring to compositions, the scope of the methods of use disclosure also includes combining more than one input (i.e. composition) for the treatment of a hydrogen system or medium in need of biofouling control.

The methods of use include adding a biocidal effective amount of the saturated alkyl quaternary ammonium compound containing compositions, aldehyde containing compositions, namely glutaraldehyde containing compositions, and/or tetrakis(hydroxymethyl)phosphonium sulfate containing compositions to a hydrogen system or medium and inhibiting biofouling therein. Beneficially, the composition reduces microbial populations compared to an environment that does not contain the saturated alkyl quaternary ammonium compound containing compositions, aldehyde containing compositions, namely glutaraldehyde containing compositions, and/or tetrakis(hydroxymethyl)phosphonium sulfate containing compositions. For example, the composition provides improved or at least the same biocidal efficacy within a system treated with a conventional biocide.

The method comprises adding a biocidal effective amount of the saturated alkyl quaternary ammonium compound containing compositions, aldehyde containing compositions, namely glutaraldehyde containing compositions, and/or tetrakis(hydroxymethyl)phosphonium sulfate containing compositions. The dosage amounts of the compositions described herein to be added to a hydrogen system or medium can be tailored by one skilled in the art based on factors for each system, including, for example, content of fluid (including gas), volume of the fluid (including gas), surface area of the system, temperatures, pH, and CO₂ content. In embodiments, an effective amount of the composition is from about 1 ppm to about 5,000 ppm, from about 1 ppm to about 1,000 ppm, or preferably from about 5 ppm to about 1,000 ppm.

In some embodiments, the biocidal effective amount of the composition is provided as a shock treatment employing a greater concentration of the composition compared to a maintenance treatment. In embodiments providing a shock treatment of dosing of the biocidal composition from about 1 ppm to about 1,000 ppm, or preferably from about 5 ppm to about 1,000 ppm, based on the total volume of the system, are provided. In embodiments providing a maintenance treatment of dosing of the biocidal composition from about 1 ppm to about 500 ppm, or preferably from about 5 ppm to about 500 ppm, based on the total volume of the system, are provided.

The compositions are applied to fluids in a hydrogen system at varying pH ranges. In an embodiment the pH of the fluids will be between about 2 and about 8.

The compositions can be applied to a fluid in a hydrogen system at any selected temperature, such as ambient temperature or an elevated temperature. The fluid (e.g., hydrogen gas) can be at a temperature of from about −250° C. to about 300° C. (including cryogenic conditions), or about −20° C. to about 85° C.

Biofouling control or biocidal efficacy provides a beneficial reduction in microbes (also referred to as a microbial population). The reduction in microbes are referred to in the methods described as reducing a microbial population, and includes, but is not limited to bacteria, viruses, fungi, and algae. Beneficially the reducing of the microbial population is on the treated surface or within the hydrogen system or medium. The reduction can take please within a few minutes. In some embodiments, the reduction takes place within about 10 minutes or less, about 5 minutes or less, or about 1 minute or less. Preferably, the reduction in microbes is at least about a 3 log, 3.5 log, 4 log, 4.5 log, or 5 log reduction of a microbial population. The biocidal efficacy can be assessed by any suitable method, including for example, microbiological, chemical, molecular, and/or biofilm assessment methods, either in-situ or ex-situ. Some examples of methods include for example, ATP (adenosine triphosphate), MPN (most probable number), plate counts, hydrogen sulfide detection, sulfate reduction test, PCR (polymerase chain reaction), FISH (fluorescent in-situ hybridization), biofilm thickness, swabs, coupons, in-line sensors, etc.

### EMBODIMENTS

The present disclosure is further defined by the following numbered embodiments:

1. A method of inhibiting corrosion in a hydrogen system comprising: contacting a surface comprising metal in a hydrogen system or medium with a corrosion-inhibiting composition comprising a saturated alkyl diquaternary ammonium compound having the following structure (III) or a saturated alkyl quaternary ammonium compound having the following structure (IV):

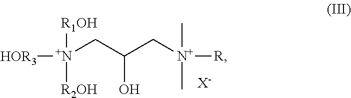

wherein: R is a linear C₈-C₃₀ alkyl group, R₁, R₂, and R₃ are independently a linear C₁-C₃ alkyl group, and X— is a halide;

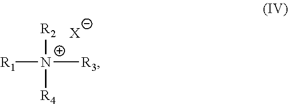

wherein: R₁, R₂, R₃ and R₄ are independently a linear C₁₋₂₄ alkyl group, and X— is a halide; and reducing corrosion on the surface.

2. The method of embodiment 1, wherein the hydrogen system or medium comprises hydrogen gas, natural gas, hydrogen sulfide gas, ammonia, or a combination thereof.

3. The method of embodiment 2, wherein the hydrogen medium comprises hydrogen gas and one or more of methane, ethane, propane, nitrogen, and/or carbon dioxide.

4. The method of any one of embodiments 1-3, wherein the saturated diquaternary ammonium compound (III) is a reaction product of

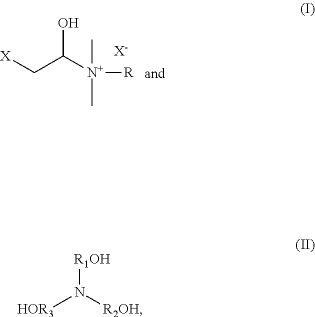

wherein R is a linear C₈-C₃₀ alkyl group and X— is a halide, and R₁, R₂, and R₃ are independently a linear C₁-C₃ alkyl group, under catalysis of a hydroxide source.

5. The method of embodiment 4, wherein the

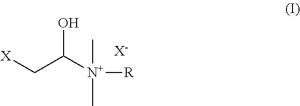

reagent is a hydrophobic modified chlorohydrin.

6. The method of any one of embodiments 1-5, wherein the saturated diquaternary ammonium compound (III) has the following structure:

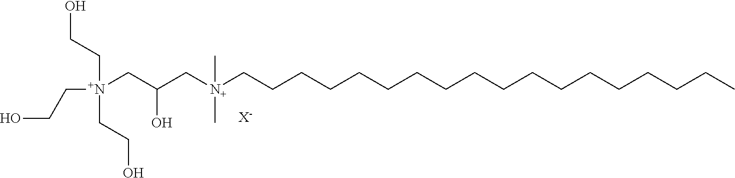

(IIIa) wherein X is Cl— or Br—.

7. The method of any one of embodiments 1-6, wherein from about 1 ppm to about 1,000 ppm based on the total volume of the system or medium of the saturated alkyl diquaternary ammonium compound is added to the hydrogen system or medium.

8. The method of embodiment 1, wherein the saturated alkyl quaternary ammonium compound (IV) has the structure

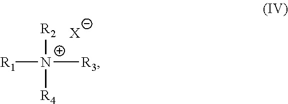

wherein: R₁ and R₂ are methyl groups, R₃ and R₄ are independently a linear C₁₀₋₂₄ alkyl group, and X— is a halide.

9. The method of embodiment 8, wherein from about 1 ppm to about 1,000 ppm based on the total volume of the system or medium of the saturated alkyl quaternary ammonium compound is added to the hydrogen system or medium.

10. The method of any one of embodiments 1-9, wherein the composition further comprises a solvent comprising water, an organic solvent, aromatic solvent, or combination thereof.

11. The method of any one of embodiments 1-10, wherein the composition further comprises at least one additional component selected from the group consisting of sulfur-containing agent, additional corrosion inhibitors, surfactants, polymers, pH modifiers, asphaltene inhibitors, paraffin inhibitors, scale inhibitors, metal complexing agents (chelants), emulsifiers, water clarifiers, dispersants, emulsion breakers, or combinations thereof.

12. The method of any one of embodiments 1-11, wherein the contacting is a dosing in a batch or continuous application.

13. The method of any one of embodiments 1-12, wherein the metal surface comprises steel.

14. The method of embodiment 13, wherein the surface is a containment used in the production, transportation, storage and/or separation of hydrogen gas.

15. The method of embodiment 14, wherein the transportation includes onshore or offshore hydrogen transportation.

16. The method of any one of embodiments 1-15, wherein the compositions are free of chemistries with unsaturated carbon-carbon double or triple bonds.

17. A method of cleaning and/or preventing biofouling in a hydrogen system comprising: contacting a surface in a hydrogen system or medium with a cleaning and/or biocidal composition comprising a saturated alkyl diquaternary ammonium compound, a saturated alkyl quaternary ammonium compound, an aldehyde comprising glutaraldehyde, formaldehyde or glyoxal, and/or tetrakis(hydroxymethyl)phosphonium sulfate; wherein the saturated alkyl diquaternary ammonium compound has the following structure (III) or a saturated alkyl quaternary ammonium compound having the following structure (IV):

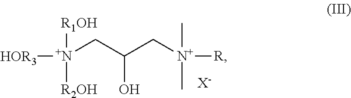

wherein: R is a linear C₈-C₃₀ alkyl group, R₁, R₂, and R₃ are independently a linear C₁-C₃ alkyl group, and X— is a halide;

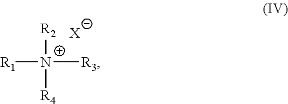

wherein: R₁, R₂, R₃ and R₄ are independently a linear C₁₋₂₄ alkyl group, and X— is a halide; and reducing a microbial population on the surface or in the hydrogen system or medium.

18. The method of embodiment 17, wherein the hydrogen system or medium comprises hydrogen gas, natural gas, hydrogen sulfide gas, ammonia, or a combination thereof.

19. The method of embodiment 18, wherein the hydrogen medium comprises hydrogen gas and one or more of methane, ethane, propane, nitrogen, and/or carbon dioxide.

20. The method of any one of embodiments 17-19, wherein the saturated diquaternary ammonium compound (III) is a reaction product of

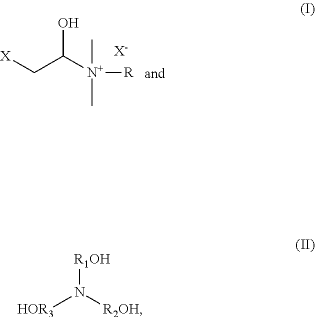

wherein R is a linear C₈-C₃₀ alkyl group and X— is a halide, and R₁, R₂, and R₃ are independently a linear C₁-C₃ alkyl group, under catalysis of a hydroxide source.

21. The method of embodiment 20, wherein the

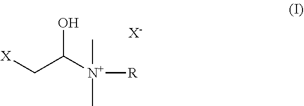

reagent is a hydrophobic modified chlorohydrin.

22. The method of any one of embodiments 17-21, wherein the saturated diquaternary ammonium compound (III) has the following structure:

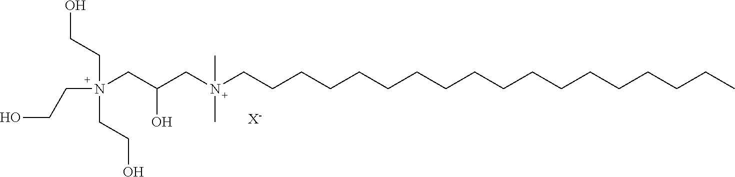

(IIIa) wherein X is Cl— or Br—.

23. The method of any one of embodiments 17-19, wherein the saturated alkyl quaternary ammonium compound (IV) has the structure

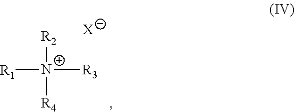

wherein: R₁ and R₂ are methyl groups, R₃ and R₄ are independently a linear C₁₀₋₂₄ alkyl group, and X— is a halide.

24. The method of any one of embodiments 20-23, wherein from about 1 ppm to about 1,000 ppm based on the total volume of the system or medium of the saturated alkyl quaternary ammonium compound or the saturated alkyl diquaternary ammonium compound is added to the hydrogen system or medium.

25. The method of any one of embodiments 17-19, wherein the cleaning and/or biocidal composition comprises the tetrakis(hydroxymethyl)phosphonium sulfate, and wherein from about 5 ppm to about 1,000 ppm of the tetrakis(hydroxymethyl)phosphonium sulfate is added to the hydrogen system or medium.

26. The method of any one of embodiments 17-19, wherein the cleaning and/or biocidal composition comprises the aldehyde comprising glutaraldehyde, formaldehyde or glyoxal, and wherein from about 5 ppm to about 1,000 ppm of the aldehyde is added to the hydrogen system or medium.

27. The method of any one of embodiments 17-26, wherein the composition further comprises a solvent comprising water, an organic solvent, aromatic solvent, or combination thereof.

28. The method of any one of embodiments 17-27, wherein the composition further comprises at least one additional component selected from the group consisting of sulfur-containing agent, additional corrosion inhibitors, surfactants, polymers, pH modifiers, asphaltene inhibitors, paraffin inhibitors, scale inhibitors, metal complexing agents (chelants), emulsifiers, water clarifiers, dispersants, emulsion breakers, or combinations thereof.

29. The method of any one of embodiments 17-28, wherein the contacting is a dosing in a batch or continuous application.

30. The method of any one of embodiments 17-29, wherein the surface is a containment used in the production, transportation, storage and/or separation of hydrogen gas.

31. The method of embodiment 30, wherein the transportation includes onshore or offshore hydrogen transportation.

32. The method of any one of embodiments 17-31, wherein the compositions are free of chemistries with unsaturated carbon-carbon double or triple bonds.

### EXAMPLES

Embodiments of the present disclosure are further defined in the following non-limiting Examples. It should be understood that these Examples, while indicating certain embodiments of the disclosure, are given by way of illustration only. From the above discussion and these Examples, one skilled in the art can ascertain the essential characteristics of this disclosure, and without departing from the spirit and scope thereof, can make various changes and modifications of the embodiments of the disclosure to adapt it to various usages and conditions. Thus, various modifications of the embodiments of the disclosure, in addition to those shown and described herein, will be apparent to those skilled in the art from the foregoing description. Such modifications are also intended to fall within the scope of the appended claims.

### Example 1

Examples were conducted to assess the corrosion inhibition of a saturated alkyl diquaternary compounds of structure (IIIa) as described above using a bubble cell test to assess corrosion performance using linear polarization resistance testing. The corrosion inhibitor (CI) was evaluated without combination of any other chemistries or formulation components.

The corrosion rate was measured by Linear Polarization Resistance (LPR) techniques. The working electrode used was carbon steel (C1018 grade). The counter and reference electrodes were both 1018 carbon steel. The electrodes were all cleaned and polished prior to testing.

The test conditions included: 80° C., CO₂ saturated fluids with 3% NaCl brine (100% brine) with continuous CO₂ sparge, atmospheric pressure. A pre-corrosion time (i.e. with no corrosion inhibitor) was carried out for about 3 hours before 500 ppm of a 20% active chemistry in solvent blend was added. This is equivalent to 100 ppm of the active chemistry being introduced into the test cell. The inhibited corrosion rate at about 15 hours after CI chemical injection was noted and a percentage inhibition determined by comparing with the corrosion rate of a carbon steel electrode under otherwise the same conditions in the absence of chemical corrosion inhibitor after the same time of exposure to the corrosive environment. The results are summarized in Table 1.

TABLE 1

| | | | CI | Corrosion | |
|---|---|---|---|---|---|
| | CI | | Active | rate after | |
| | Dosage | | Injected | 15 hour CI | |
| | activity | Dosage | in Test | injection | % |
| | (%) | (ppm) | (ppm) | (mpy) | Protection |
| CI Structure IIIa | 20 | 500 | 100 | 2 | >99 |

The results show the use of saturated dialkyl quaternary ammonium compounds of structures III, namely IIIa as evaluated provides significant corrosion inhibition under the hydrogen-rich environment conditions evaluated. Beneficially the structure of the saturated dialkyl quaternary ammonium compounds will prevent hydrogenation leading to CI instability and performance issues in hydrogen rich environments providing a benefit over conventional unsaturated corrosion inhibitors.

It is to be understood that while the invention has been described in conjunction with the detailed description thereof, the foregoing description is intended to illustrate, and not limit the scope of the invention, which is defined by the scope of the appended claims. Other embodiments, advantages, and modifications are within the scope of the following claims. Any reference to accompanying drawings which form a part hereof, are shown, by way of illustration only. It is understood that other embodiments may be utilized and structural changes may be made without departing from the scope of the present disclosure. All publications discussed and/or referenced herein are incorporated herein in their entirety.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be utilized for realizing the invention in diverse forms thereof.

What is claimed is:

1. A method of inhibiting corrosion in a hydrogen system comprising:
contacting a surface comprising metal in a hydrogen system or medium with a corrosion-inhibiting composition comprising a saturated alkyl diquaternary ammonium compound having the following structure (III) or a saturated alkyl quaternary ammonium compound having the following structure (IV): 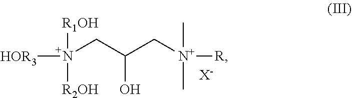
wherein:
R is a linear C₈-C₃₀ alkyl group,
R₁, R₂, and R₃ are independently a linear C₁-C₃ alkyl group, and
X— is a halide; 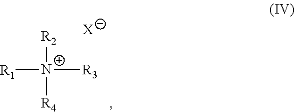
wherein:
R₁, R₂, R₃ and R₄ are independently a linear C₁₋₂₄ alkyl group, and X— is a halide; and
reducing corrosion on the surface.

2. The method of claim 1, wherein the hydrogen system or medium comprises hydrogen gas, natural gas, hydrogen sulfide gas, ammonia, or a combination thereof.

3. The method of claim 2, wherein the hydrogen medium comprises hydrogen gas and one or more of methane, ethane, propane, nitrogen, and/or carbon dioxide.

4. The method of claim 1, wherein the saturated diquaternary ammonium compound (III) is a reaction product of 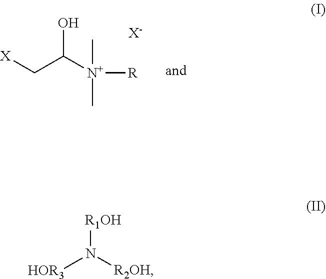
wherein R is a linear C₈-C₃₀ alkyl group and X— is a halide, and R₁, R₂, and R₃ are independently a linear C₁-C₃ alkyl group, under catalysis of a hydroxide source.

5. The method of claim 4, wherein the 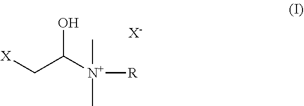
reagent is a hydrophobic modified chlorohydrin.

6. The method of claim 1, wherein the saturated diquaternary ammonium compound (III) has the following structure: 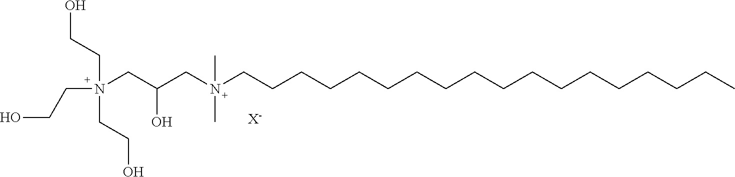
(IIIa) wherein X is Cl— or Br—.

7. The method of claim 1, wherein from about 1 ppm to about 1,000 ppm based on the total volume of the system or medium of the saturated alkyl diquaternary ammonium compound is added to the hydrogen system or medium.

8. The method of claim 1, wherein the saturated alkyl quaternary ammonium compound (IV) has the structure 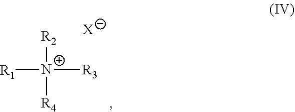
wherein: R₁ and R₂ are methyl groups, R₃ and R₄ are independently a linear C₁₀₋₂₄ alkyl group, and X— is a halide.

9. The method of claim 8, wherein from about 1 ppm to about 1,000 ppm based on the total volume of the system or medium of the saturated alkyl quaternary ammonium compound is added to the hydrogen system or medium.

10. The method of claim 1, wherein the composition further comprises a solvent comprising water, an organic solvent, aromatic solvent, or combination thereof.

11. The method of claim 1, wherein the composition further comprises at least one additional component selected from the group consisting of sulfur-containing agent, additional corrosion inhibitors, surfactants, polymers, pH modifiers, asphaltene inhibitors, paraffin inhibitors, scale inhibitors, metal complexing agents, emulsifiers, water clarifiers, dispersants, emulsion breakers, or combinations thereof.

12. The method of claim 1, wherein the contacting is a dosing in a batch or continuous application.

13. The method of claim 1, wherein the metal surface comprises steel.

14. The method of claim 13, wherein the surface is a containment used in the production, transportation, storage and/or separation of hydrogen gas.

15. The method of claim 14, wherein the transportation includes onshore or offshore hydrogen transportation.

16. The method of claim 1, wherein the compositions are free of chemistries with unsaturated carbon-carbon double or triple bonds.

17. A method of cleaning and/or preventing biofouling in a hydrogen system comprising:
contacting a surface in a hydrogen system or medium with a cleaning and/or biocidal composition comprising a saturated alkyl diquaternary ammonium compound, a saturated alkyl quaternary ammonium compound, an aldehyde comprising glutaraldehyde, formaldehyde or glyoxal, and/or tetrakis(hydroxymethyl)phosphonium sulfate;
wherein the saturated alkyl diquaternary ammonium compound has the following structure (III) or a saturated alkyl quaternary ammonium compound having the following structure (IV): 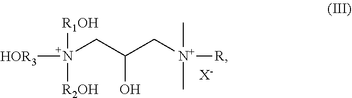
wherein:
R is a linear C₈-C₃₀ alkyl group,
R₁, R₂, and R₃ are independently a linear C₁-C₃ alkyl group, and
X— is a halide; 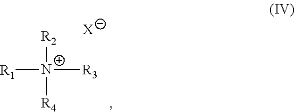
wherein:
R₁, R₂, R₃ and R₄ are independently a linear C₁₋₂₄ alkyl group, and X— is a halide; and
reducing a microbial population on the surface or in the hydrogen system or medium.

18. The method of claim 17, wherein the hydrogen system or medium comprises hydrogen gas, natural gas, hydrogen sulfide gas, ammonia, or a combination thereof.

19. The method of claim 18, wherein the hydrogen medium comprises hydrogen gas and one or more of methane, ethane, propane, nitrogen, and/or carbon dioxide.

20. The method of claim 17, wherein the saturated diquaternary ammonium compound (III) is a reaction product of 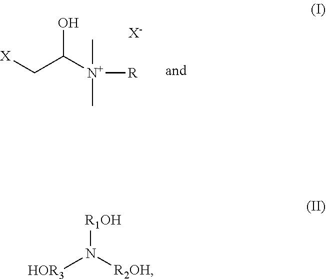
wherein R is a linear C₈-C₃₀ alkyl group and X— is a halide, and R₁, R₂, and R₃ are independently a linear C₁-C₃ alkyl group, under catalysis of a hydroxide source.

21. The method of claim 20, wherein the 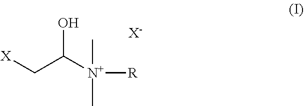
reagent is a hydrophobic modified chlorohydrin.

22. The method of claim 17, wherein the saturated diquaternary ammonium compound (III) has the following structure: 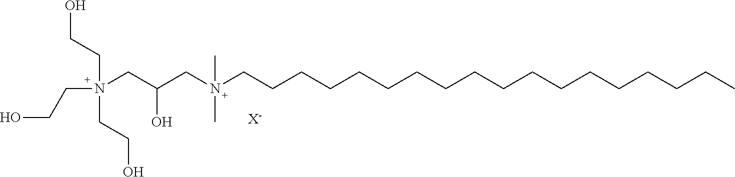
(IIIa) wherein X is Cl— or Br—.

23. The method of claim 17, wherein the saturated alkyl quaternary ammonium compound (IV) has the structure 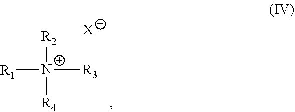
wherein: R₁ and R₂ are methyl groups, R₃ and R₄ are independently a linear C₁₀₋₂₄ alkyl group, and X— is a halide.

24. The method of claim 20, wherein from about 1 ppm to about 1,000 ppm based on the total volume of the system or medium of the saturated alkyl quaternary ammonium compound or the saturated alkyl diquaternary ammonium compound is added to the hydrogen system or medium.

25. The method of claim 17, wherein the cleaning and/or biocidal composition comprises the tetrakis(hydroxymethyl)phosphonium sulfate, and wherein from about 5 ppm to about 1,000 ppm of the tetrakis(hydroxymethyl)phosphonium sulfate is added to the hydrogen system or medium.

26. The method of claim 17, wherein the cleaning and/or biocidal composition comprises the aldehyde comprising glutaraldehyde, formaldehyde or glyoxal, and wherein from about 5 ppm to about 1,000 ppm of the aldehyde is added to the hydrogen system or medium.

27. The method of claim 17, wherein the composition further comprises a solvent comprising water, an organic solvent, aromatic solvent, or combination thereof.

28. The method of claim 17, wherein the composition further comprises at least one additional component selected from the group consisting of sulfur-containing agent, additional corrosion inhibitors, surfactants, polymers, pH modifiers, asphaltene inhibitors, paraffin inhibitors, scale inhibitors, metal complexing agents, emulsifiers, water clarifiers, dispersants, emulsion breakers, or combinations thereof.

29. The method of claim 17, wherein the contacting is a dosing in a batch or continuous application.

30. The method of claim 17, wherein the surface is a containment used in the production, transportation, storage and/or separation of hydrogen gas.

31. The method of claim 30, wherein the transportation includes onshore or offshore hydrogen transportation.

32. The method of claim 17, wherein the compositions are free of chemistries with unsaturated carbon-carbon double or triple bonds.
